(19) United States
(12) Patent Application Publication
(10) Pub. No.: US 2015/0087986 A1
(43) Pub. Date: Mar. 26, 2015

(54) SYSTEMS AND METHODS FOR PRODUCING INTRAVASCULAR IMAGES

(71) Applicant: Volcano Corporation, San Diego, CA (US)

(72) Inventors: Anuja Nair, Bedford, MA (US); Andrew Hancock, Sacramento, CA (US); Shukui Zhao, Folsom, CA (US)

(73) Assignee: VOLCANO CORPORATION, San Diego, CA (US)

(21) Appl. No.: 14/037,683

(22) Filed: Sep. 26, 2013

(51) Int. Cl. A61B 8/12

(52) U.S. Cl. CPC A61B 8/12
USPC 600/447

(57) ABSTRACT

The invention generally relates to systems and methods for producing intravascular images. In certain embodiments, systems and methods of the invention involve causing transducers of an intravascular ultrasound device to generate a plurality of different types of data, each type of data being based on a different manner of operation of the transducers. The systems and methods of the invention additionally involve adjusting the manner of operation of the transducers for one of the types of data to thereby adjust an acquisition frame rate of the one type of data to an adjusted acquisition frame rate. The one type of data at the adjusted acquisition frame rate is then received and an intravascular image that includes the one type of data at the adjusted acquisition frame rate is displayed.

### FIELD OF THE INVENTION

The invention generally relates to systems and methods for producing intravascular images.

### BACKGROUND

Intravascular Ultrasound (IVUS) is an important interventional diagnostic modality for imaging atherosclerosis and other vascular diseases and defects. In the procedure, an IVUS catheter is threaded over a guidewire into a blood vessel, and images of atherosclerotic plaque and surrounding areas are acquired using ultrasonic echoes.

There are two types of IVUS catheters commonly in use, mechanical/rotational IVUS catheters and solid state catheters. A solid state catheter (or phased array) has no rotating parts, but instead includes an array of transducer elements. The same transducer elements can be used to produce different types of intravascular data, based on the manner in which the transducer elements operate. For example, the same transducer array may be used to generate intravascular structural-image data and to generate flow data by changing the operation of the elements.

A problem with using a single set of transducer elements to acquire multiple types of data in this manner is that the acquisition frame rate from any one type of data is decreased when the transducers operate in dual or multiple imaging mode. For example, for a dual imaging case, when the transducers are only acquiring image data, the data can be acquired at about 20 to 30 frames per second for higher frequency devices. However, if that same set of transducers is used to acquire both image and flow data, and the image and flow data are acquired in different ways, the acquisition frame rate for the image data drops to as low as 12 to 15 frames per second. For IVUS catheters that generate image data at a rate of 10 frames per second (e.g. lower frequency devices), adding flow functionality drops the frame rate to as to as low as 4 to 5 frames per second. Lowering the acquisition frame rate increases the sampling time to collect a frame and results in lower time-resolution images, which increases the likelihood that an operator will miss a defect in a vessel or implanted intravascular device, such as a stent.

### SUMMARY

The invention provides systems and methods that compensate for a decrease in acquisition frame rate when a single set of intravascular ultrasound (IVUS) transducer elements is used to acquire more than one set of intravascular data that are different for each imaging mode. Aspects of the invention are accomplished by modifying the manner in which the IVUS transducer elements operate to compensate for the overall decrease in acquisition frame rate that occurs when a single set of IVUS transducer elements operate in dual acquisition mode.

In certain aspects, the invention provides systems for producing an intravascular image that includes a central processing unit (CPU) and storage coupled to the CPU for storing instructions. The instructions, when executed by the CPU, cause transducers in an intravascular ultrasound device to generate a plurality of different types of data, each type of data being based on a different manner of transducer operation. The instructions additionally cause the CPU to adjust its manner of operation with respect to one of the types of data in order to adjust an acquisition frame rate. The CPU is thus able to receive one type of data at the adjusted acquisition frame rate, and display an intravascular image that includes such data.

In certain aspects, the invention provides methods for producing an intravascular image that involve causing transducers to generate a plurality of different types of data, each type of data being based on a different manner of transducer operation. The methods additionally involve adjusting the manner of operation of the transducers for one of the types of data in order to adjust an acquisition frame rate with respect to that type of data. The methods additionally involve receiving the adjusted frame rate data and displaying an intravascular image that comprises the adjusted frame rate data.

There are numerous techniques for adjusting transducer operation. For example, adjusting the manner of operation of the transducers may be performed by decreasing the imaging field-of-view of the transducers for one type of data. Decreasing the field-of-view for any one type of data shortens each transmit/receive sequence for that type of data. Shortening the transmit/receive sequence allows for faster data acquisition, allowing the transducers to more quickly switch from acquiring such data to acquiring any other type of data. Such an approach increases the acquisition frame rate for not only the adjusted data type, but also the unadjusted types of data.

In another embodiment, adjusting the manner in which the transducers operate involves revising the manner of operation of the transducers to lower the acquisition resolution of one type of data. An example of this embodiment involves revising the transmit/receive sequence to decrease the number of acquisitions in order to move more quickly move around a catheter body to produce 360 degrees of information from within a vascular structure. For example, a traditional acquisition sequence for acquiring flow data involves firing four adjacent transducer elements simultaneously and then receiving on the same four elements simultaneously. That sequence is repeated 64 times on the same four elements, producing 32 averaged A scan-lines of data from the same physical beam location. The sequence moves over one transducer element to form the next set of four transducer elements. The process is then repeated on the next set of four elements. In the traditional transmit/receive sequence, no transducer elements are skipped between different transducer sets. An adjusted transmit/receive sequence would involve skipping over one or more transducer elements when forming a new set, i.e., move two or more transducers from the previous set of four to form the next set of four transducers (or apertures). Decreasing the number of sets allows for faster completion of the cycle around the catheter body, which results in faster acquisition of the adjusted type of data for that one 360 degree frame of data. That allows the transducers to more quickly switch from acquiring the adjusted data to acquiring any other type of data, thereby increasing the acquisition frame rate for not only the adjusted data, but also the display of the composite data.

Adjusting the manner of operation of the transducers may also be performed by limiting the transmit/receive of one, some, or all imaging modes to a particular sector in the 360 degree typical IVUS data. Such an approach involves selecting a sub-set of the transducers to be used to acquire one, some or all types of data from a sub-region of the inner vascular circumference. In that manner, a subset smaller than the full set of all of the transducers elements need to be used to generate the one, some or all types of data that corresponds to the circumferential area of interest. Accordingly, the acquisition frame rate for the one, some or all types of data has been increased, and because the transducers spend less time generating all the types of data, the acquisition frame rate for the display of the composite data from these types of data also increases.

In another embodiment, the sector approach involves using the transducers to produce a 360° intravascular image, displaying the 360° intravascular image, selecting an area-of-interest within the image, and selecting a sub-set of the transducers to acquire the one type of data in the area-of-interest. This embodiment allows correct selection of the area-of interest from the full data set. In another embodiment of the sector approach, the previous embodiment produces a lower resolution 360° intravascular image. Another embodiment of the sector approach involves using the transducers to produce a 360° intravascular image, displaying the 360° intravascular image, selecting multiple areas-of-interest within the image, selecting multiple sub-sets of transducers, each sub-set of transducers corresponding to a selected area-of interest, and using the sub-sets of transducers to acquire the one type of data in each of the areas-of-interests. In any of those embodiments, selecting may be done manually by an operator or automatically by the system, or a combination of the two.

Typically, although not required, the produced intravascular image further includes at least one other type of data. In an exemplary embodiment, a first type of data is intravascular structural-image data and a second type of data is intravascular flow data. The acquisition of either or both may be adjusted to increase the overall acquisition frame rate when the transducers are operating in dual acquisition mode. In certain embodiments, the intravascular flow data is the one type of data that is adjusted. In other embodiments, the intravascular structural-image data is the one type of data that is adjusted. In other embodiments, both the intravascular flow and structural-image data are adjusted.

### BRIEF DESCRIPTION OF THE DRAWINGS

FIG. 1 is a schematic drawing showing an illustrative embodiment of an IVUS medical system in a catheterization laboratory.

FIG. 2 is a schematic drawing showing an IVUS catheter

FIG. 3 is a schematic drawing showing a set of array transducer elements operating in traditional gray-scale structural-imaging mode.

FIG. 4 is a graph showing a typical individual A scan line with Time on the x-axis that corresponds to distance from the catheter into the vessel lumen and wall.

FIG. 5 depicts a scan-converted and log-compressed gray-scale 360 degree IVUS image constructed from multiple A scan-lines. Each of the dotted lines on the image represents one scan line.

FIG. 6 is a schematic drawing showing a set of array transducer elements operating in flow imaging mode.

FIG. 7 panel A is a gray-scale IVUS image of a vessel. FIG. 7 panel B is an image of flow within the vessel. FIG. 7 panel C is a composite image of the flow data overlaid on the gray-scale image.

### DETAILED DESCRIPTION

The invention generally relates to systems and methods for producing intravascular images from two different types of data acquired from an intravascular ultrasound (IVUS) device. IVUS imaging is widely used in interventional cardiology as a diagnostic tool for assessing a diseased vessel, such as an artery, within the human body to determine the need for treatment, to guide an intervention, and/or to assess its effectiveness. An IVUS device including one or more ultrasound transducers is introduced into the vessel and guided to the area to be imaged. The transducers emit and then receive backscattered ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a 360 degree cross-sectional image of the vessel where the device is placed.

There are two general types of IVUS devices in use today: rotational and solid-state (also known as synthetic aperture phased array). For a typical rotational IVUS device, a single ultrasound transducer element is located at the tip of a flexible driveshaft that spins inside a plastic sheath inserted into the vessel of interest. The transducer element is oriented such that the ultrasound beam propagates generally perpendicular to the axis of the device. The fluid-filled sheath protects the vessel tissue from the spinning transducer and driveshaft while permitting ultrasound signals to propagate from the transducer into the tissue and back. As the driveshaft rotates, the transducer is periodically excited with a high voltage pulse to emit a short burst of ultrasound. The same transducer then listens for the returning echoes reflected from various tissue structures. The IVUS imaging system assembles a two dimensional display of the vessel cross-section from a sequence of pulse/acquisition cycles occurring during a single revolution of the transducer.

In contrast, solid-state IVUS devices carry a transducer complex that includes an array of ultrasound transducers distributed around the circumference of the device connected to a set of transducer controllers. The transducer controllers select transducer sets for transmitting an ultrasound pulse and for receiving the echo signal. By stepping through a sequence of transmit-receive sets, the solid-state IVUS system can synthesize the effect of a mechanically scanned transducer element but without moving parts. The same transducer elements can be used to acquire different types of intravascular data. The different types of intravascular data are acquired based on different manners of operation of the transducer elements. The solid-state scanner can be wired directly to the imaging system with a simple electrical cable and a standard detachable electrical connector. While aspects of the invention are described in relation to solid-state IVUS devices, one of skill in the art will recognize that the invention also applies to rotational IVUS devices.

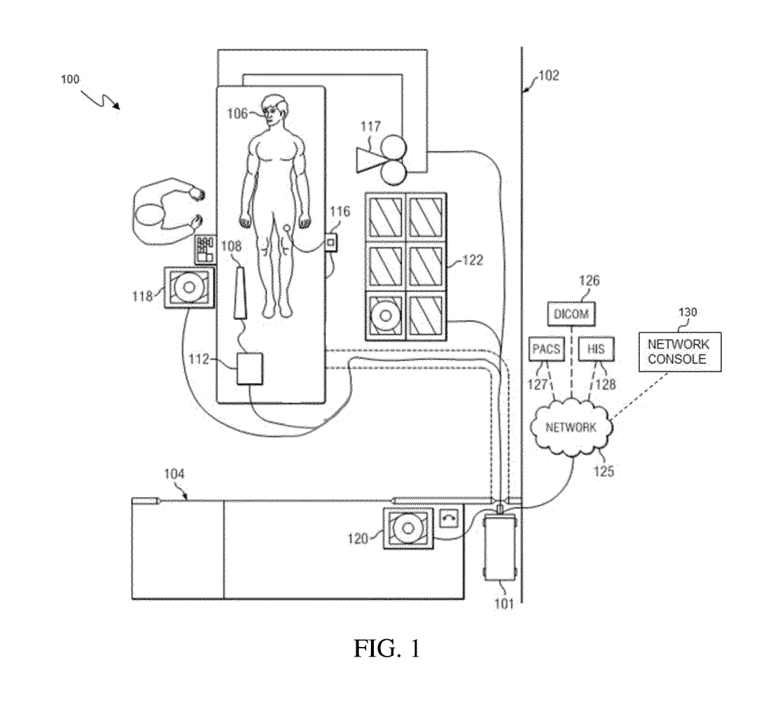

FIG. 1 is a schematic drawing depicting a medical system including an IVUS imaging system in various applications according to some embodiments of the present disclosure. In general, the medical system 100 may be a single modality medical system, such as an IVUS system, and may also be a multi-modality medical system. In that regard, a multi-modality medical system provides for coherent integration and consolidation of multiple forms of acquisition and processing elements designed to be sensitive to a variety of methods used to acquire and interpret human biological physiology and morphological information and coordinate treatment of various conditions.

With reference to FIG. 1, the imaging system 101 is an integrated device for the acquisition, control, interpretation, and display of one or more modalities of medical sensing data. Accordingly, in some embodiments, the imaging system 101 is a single modality imaging system, such as an IVUS imaging system, whereas, in some embodiments, the imaging system 101 is a multi-modality imaging system. In one embodiment, the imaging system 101 includes a computer system with the hardware and software to acquire, process, and display medical imaging data, but, in other embodiments, the imaging system 101 includes any other type of computing system operable to process medical data. In the embodiments in which the imaging system 101 includes a computer workstation, the system includes a processor such as a microcontroller or a dedicated central processing unit (CPU), a non-transitory computer-readable storage medium such as a hard drive, random access memory (RAM), and/or compact disk read only memory (CD-ROM), a video controller such as a graphics processing unit (GPU), and/or a network communication device such as an Ethernet controller and/or wireless communication controller. In that regard, in some particular instances, the imaging system 101 is programmed to execute steps associated with the data acquisition and analysis described herein. Accordingly, it is understood that any steps related to data acquisition, data processing, instrument control, and/or other processing or control aspects of the present disclosure may be implemented by the imaging system 101 using corresponding instructions stored on or in a non-transitory computer readable medium accessible by the processing system. In some instances, the imaging system 101 is portable (e.g., handheld, on a rolling cart, etc.). Further, it is understood that in some instances imaging system 101 includes a plurality of computing devices. In that regard, it is particularly understood that the different processing and/or control aspects of the present disclosure may be implemented separately or within predefined groupings using a plurality of computing devices. Any divisions and/or combinations of the processing and/or control aspects described below across multiple computing devices are within the scope of the present disclosure.

In the illustrated embodiment, the medical system 100 is deployed in a catheter lab 102 having a control room 104, with the imaging system 101 being located in the control room. In other embodiments, the imaging system 101 may be located elsewhere, such as in the catheter lab 102, in a centralized area in a medical facility, or at an off-site location accessible over a network. For example, the imaging system 101 may be a cloud-based resource. The catheter lab 102 includes a sterile field generally encompassing a procedure area, whereas the associated control room 104 may or may not be sterile depending on the requirements of a procedure and/or health care facility. The catheter lab and control room may be used to perform on a patient any number of medical sensing procedures such as intravascular ultrasound (IVUS), angiography, virtual histology (VH), forward looking IVUS (FL-IVUS), intravascular photoacoustic (IVPA) imaging, a fractional flow reserve (FFR) determination, a coronary flow reserve (CFR) determination, optical coherence tomography (OCT), computed tomography (CT), intracardiac echocardiography (ICE), forward-looking ICE (FLICE), intravascular palpography, transesophageal ultrasound (TEE), thermography, magnetic resonance imaging (MRI), micro-magnetic resonance imaging (mMRI or μMRI), or any other medical sensing modalities known in the art. Further, the catheter lab and control room may be used to perform one or more treatment or therapy procedures on a patient such as radiofrequency ablation (RFA), cryotherapy, atherectomy or any other medical treatment procedure known in the art. For example, in catheter lab 102 a patient 106 may be undergoing a multi-modality procedure either as a single procedure or multiple procedures. In any case, the catheter lab 102 includes a plurality of medical instruments including medical sensing devices that collects medical sensing data in various different medical sensing modalities from the patient 106.

In the illustrated embodiment of FIG. 1, instrument 108 is a medical sensing device that may be utilized by a clinician to acquire medical sensing data about the patient 106. For instance, the instrument may collect one of pressure, flow (velocity), images (including images obtained using ultrasound (e.g., IVUS), OCT, thermal, and/or other imaging techniques), temperature, and/or combinations thereof. In some embodiments, device 108 collects medical sensing data in different versions of similar modalities. For example, in one such embodiment, device 108 collects pressure data and image data. In another such embodiment, device 108 collects 10 MHz IVUS data, 20 MHz IVUS data, or 40 MHz IVUS data. Accordingly, the device 108 may be any form of device, instrument, or probe sized and shaped to be positioned within a vessel, attached to an exterior of the patient, or scanned across a patient at a distance.

In the illustrated embodiment of FIG. 1, instrument 108 is an IVUS catheter 108 that may include one or more sensors such as a phased-array transducer to collect IVUS sensing data. In some embodiments, the IVUS catheter 108 may be capable of multi-modality sensing such as image and flow sensing. In some instances, an IVUS patient interface module (PIM) 112 is coupled to the IVUS catheter 108, which is coupled to the imaging system 101. In particular, the IVUS PIM 112 is operable to receive medical sensing data collected from the patient 106 by the IVUS catheter 108 and is operable to transmit the received data to the imaging system 101 in the control room 104. In one embodiment, the PIM 112 includes analog to digital (A/D) converters and transmits digital data to the imaging system 101. However, in other embodiments, the PIM transmits analog data to the processing system. In one embodiment, the IVUS PIM 112 transmits the medical sensing data over a Peripheral Component Interconnect Express (PCIe) data bus connection, but, in other embodiments, it may transmit data over a USB connection, a Thunderbolt connection, a FireWire connection, an Ethernet connection, or some other high-speed data bus connection. In other instances, the PIM may be connected to the imaging system 101 via wireless connections using IEEE 802.11 Wi-Fi standards, Ultra Wide-Band (UWB) standards, wireless FireWire, wireless USB, or another high-speed wireless networking standard.

Additionally, in the medical system 100, an electrocardiogram (ECG) device 116 is operable to transmit electrocardiogram signals or other hemodynamic data from patient 106 to the imaging system 101. Further, an angiogram system 117 is operable to collect x-ray, computed tomography (CT), or magnetic resonance images (MRI) of the patient 106 and transmit them to the imaging system 101. In one embodiment, the angiogram system 117 is communicatively coupled to the processing system of the imaging system 101 through an adapter device. Such an adaptor device may transform data from a proprietary third-party format into a format usable by the imaging system 101. In some embodiments, the imaging system 101 is operable to co-register image data from angiogram system 117 (e.g., x-ray data, MRI data, CT data, etc.) with sensing data from the IVUS catheter 108. As one aspect of this, the co-registration may be performed to generate three- and four-dimensional images with the sensing data.

A bedside controller 118 is also communicatively coupled to the imaging system 101 and provides user control of the particular medical modality (or modalities) being used to diagnose the patient 106. In the current embodiment, the bedside controller 118 is a touch screen controller that provides user controls and diagnostic images on a single surface. In alternative embodiments, however, the bedside controller 118 may include both a non-interactive display and separate controls such as physical buttons and/or a joystick. In the integrated medical system 100, the bedside controller 118 is operable to present workflow control options and patient image data in graphical user interfaces (GUIs). In some embodiments, the bedside controller 118 includes a user interface (UI) framework service through which workflows associated with multiple modalities may execute. Thus, the bedside controller 118 may be capable of displaying workflows and diagnostic images for multiple modalities allowing a clinician to control the acquisition of multi-modality medical sensing data with a single interface device.

A main controller 120 in the control room 104 is also communicatively coupled to the imaging system 101 and, as shown in FIG. 1, is adjacent to catheter lab 102. In the current embodiment, the main controller 120 is similar to the bedside controller 118 in that it includes a touch screen and is operable to display a multitude of GUI-based workflows corresponding to different medical sensing modalities via a UI framework service executing thereon. In some embodiments, the main controller 120 is used to simultaneously carry out a different aspect of a procedure's workflow than the bedside controller 118. In alternative embodiments, the main controller 120 includes a non-interactive display and standalone controls such as a mouse and keyboard.

The medical system 100 further includes a boom display 122 communicatively coupled to the imaging system 101. The boom display 122 may include an array of monitors, each capable of displaying different information associated with a medical sensing procedure. For example, during an IVUS procedure, one monitor in the boom display 122 may display a tomographic view and one monitor may display a sagittal view.

Further, the multi-modality imaging system 101 is communicatively coupled to a data network 125. In the illustrated embodiment, the data network 125 is a TCP/IP-based local area network (LAN); however, in other embodiments, it may utilize a different protocol such as Synchronous Optical Networking (SONET), or may be a wide area network (WAN). The imaging system 101 may connect to various resources via the network 125. For example, the imaging system 101 may communicate with a Digital Imaging and Communications in Medicine (DICOM) system 126, a Picture Archiving and Communication System (PACS) 127, and a Hospital Information System (HIS) 128 through the network 125. Additionally, in some embodiments, a network console 130 may communicate with the imaging system 101 via the network 125 to allow a doctor or other health professional to access the aspects of the medical system 100 remotely. For instance, a user of the network console 130 may access patient medical data such as diagnostic images collected by imaging system 101, or, in some embodiments, may monitor or control one or more on-going procedures in the catheter lab 102 in real-time. The network console 130 may be any sort of computing device with a network connection such as a PC, laptop, smartphone, tablet computer, or other such device located inside or outside of a health care facility.

Additionally, in the illustrated embodiment, medical sensing tools in system 100 discussed above are shown as communicatively coupled to the imaging system 101 via a wired connection such as a standard copper link or a fiber optic link, but, in alternative embodiments, the tools may be connected to the imaging system 101 via wireless connections using IEEE 802.11 Wi-Fi standards, Ultra Wide-Band (UWB) standards, wireless FireWire, wireless USB, or another high-speed wireless networking standard.

One of ordinary skill in the art would recognize that the medical system 100 described above is simply an example embodiment of a system that is operable to collect diagnostic data associated with a plurality of medical modalities. In alternative embodiments, different and/or additional tools may be communicatively coupled to the imaging system 101 so as to contribute additional and/or different functionality to the medical system 100.

Aspects of the invention are carried out using IVUS devices. Typically, IVUS devices of the invention are provided in the form of a catheter. The general design and construction of IVUS catheters is shown, for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et al., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, and other references well known in the art relating to intraluminal ultrasound devices and modalities. The catheter will typically have proximal and distal regions, and will include an imaging tip located in the distal region. Such catheters have an ability to obtain echographic images of the area surrounding the imaging tip when located in a region of interest inside the body of a patient. The catheter, and its associated electronic circuitry, will also be capable of defining the position of the catheter axis with respect to each echographic data set obtained in the region of interest.

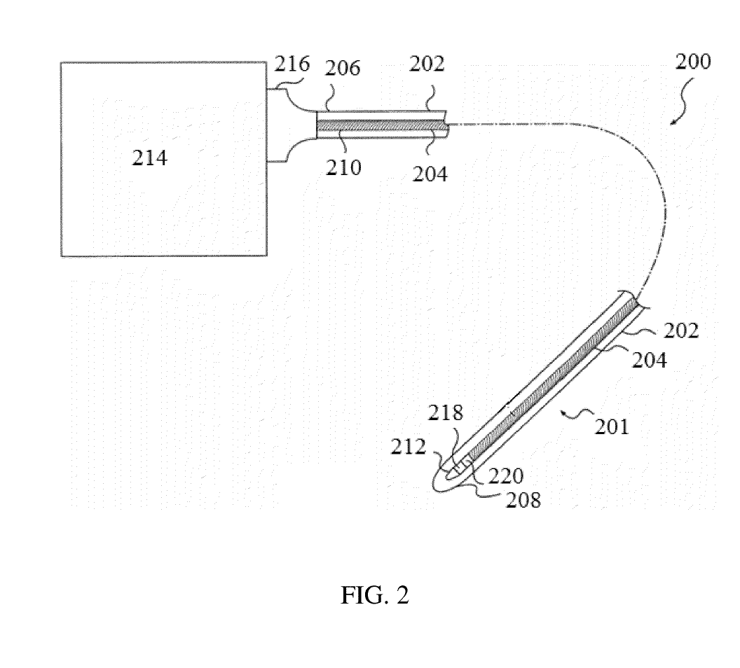

FIG. 2 shows a solid-state intravascular ultrasound probe 200 for insertion into a patient for diagnostic imaging. The probe 200 includes a catheter 201 having a catheter body 202 and a hollow transducer shaft 204. The catheter body 202 is flexible and has both a proximal end portion 206 and a distal end portion 208. The catheter body 202 may be a single lumen polymer extrusion, for example, made of polyethylene (PE), although other polymers may be used. Further, the catheter body 202 may be formed of multiple grades of PE, for example, HDPE and LDPE, such that the proximal portion exhibits a higher degree of stiffness relative to the mid and distal portions of the catheter body. This configuration provides an operator with catheter handling properties required to efficiently perform the desired procedures.

The catheter body 202 is a sheath surrounding the transducer shaft 204. For explanatory purposes, the catheter body 202 in FIG. 2 is illustrated as visually transparent such that the transducer shaft 204 disposed therein can be seen, although it will be appreciated that the catheter body 202 may or may not be visually transparent. The transducer shaft 204 may be flushed with a sterile fluid, such as saline, within the catheter body 202. A fluid injection port (not shown) may be supplied at a junction of the catheter body 202 to the interface module so that the space inside the catheter body 202 can be flushed initially and periodically. The fluid serves to eliminate the presence of air pockets around the transducer shaft 204 that adversely affect image quality. The transducer shaft 204 has a proximal end portion 210 disposed within the proximal end portion 206 of the catheter body 202 and a distal end portion 212 disposed within the distal end portion 208 of the catheter body 202.

The distal end portion 208 of the catheter body 202 and the distal end portion 212 of the transducer shaft 204 are inserted into a patient. The usable length of the probe 200 (the portion that can be inserted into a patient) can be any suitable length and can be varied depending upon the application. The distal end portion 212 of the transducer shaft 204 includes a transducer subassembly 218.

The transducer subassembly 218 is used to obtain ultrasound information from within a vessel. It will be appreciated that any suitable frequency and any suitable quantity of frequencies may be used. Exemplary frequencies range from about 5 MHz to about 80 MHz. In certain embodiments, the IVUS transducers operate at 10 MHz, or 20 MHz. Generally, lower frequency information (e.g., less than 40 MHz) facilitates a tissue versus blood classification scheme due to the strong frequency dependence of the backscatter coefficient of the blood. Higher frequency information (e.g., greater than 40 MHz) generally provides better resolution at the expense of poor differentiation between blood and tissue, which can make it difficult to identify the vessel-lumen border. Flow detection algorithms, including motion-detection algorithms (such as CHROMAFLO (IVUS fluid flow display software; Volcano Corporation), Q-Flow, B-Flow, Delta-Phase, Doppler, Power Doppler, etc.), temporal algorithms, harmonic signal processing, can be used to differentiate blood speckle from other structural tissue, and therefore enhance images where ultrasound energy back scattered from blood causes image artifacts.

The catheter body 202 may include a flexible atraumatic distal tip. For example, an integrated distal tip can increase the safety of the catheter by eliminating the joint between the distal tip and the catheter body. The integral tip can provide a smoother inner diameter for ease of tissue movement into a collection chamber in the tip. During manufacturing, the transition from the housing to the flexible distal tip can be finished with a polymer laminate over the material housing. No weld, crimp, or screw joint is usually required. The atraumatic distal tip permits advancing the catheter distally through the blood vessel or other body lumen while reducing any damage caused to the body lumen by the catheter. Typically, the distal tip will have a guidewire channel to permit the catheter to be guided to the target lesion over a guidewire. In some exemplary configurations, the atraumatic distal tip includes a coil. In some configurations the distal tip has a rounded, blunt distal end. The catheter body can be tubular and have a forward-facing circular aperture which communicates with the atraumatic tip.

The interface module 214 communicates with the transducer subassembly 218 by sending and receiving electrical signals to and from the transducer subassembly 218 via at least one electrical signal transmission member (e.g., wires or coaxial cable) within the transducer shaft 204. The interface module 214 can receive, analyze, and/or display information received through the transducer shaft 204. It will be appreciated that any suitable functionality, controls, information processing and analysis, and display can be incorporated into the interface module 214. Further description of the interface module is provided, for example in Corl (U.S. patent application number 2010/0234736).

The transducer shaft 204 includes a transducer subassembly 218 and a transducer housing 220. The transducer subassembly 218 is coupled to the transducer housing 220. The transducer housing 220 is located at the distal end portion 212 of the transducer shaft 204. The transducer subassembly 218 can be of any suitable type, including but not limited to one or more advanced transducer technologies such as PMUT or CMUT.

The transducer subassembly 218 can include either a single transducer or an array. In certain embodiments, the transducer subassembly 18 is an array of 64 individual transducer elements. The 64 transducer elements are distributed around the circumference of the transducer shaft 204 and are operably connected to the interface module 214. The interface module 214 selects transducer sets for transmitting an ultrasound pulse and for receiving the echo signal. By stepping through a sequence of transmit-receive sets, the solid-state IVUS system can synthesize the effect of a mechanically scanned transducer element but without moving parts.

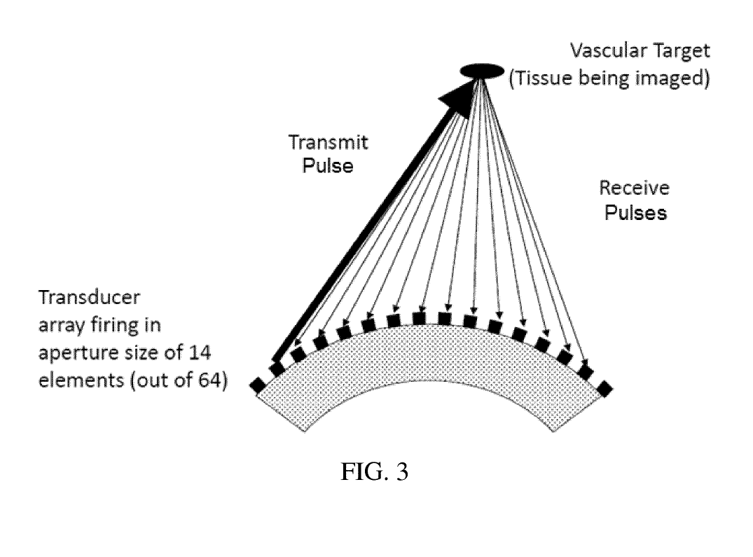

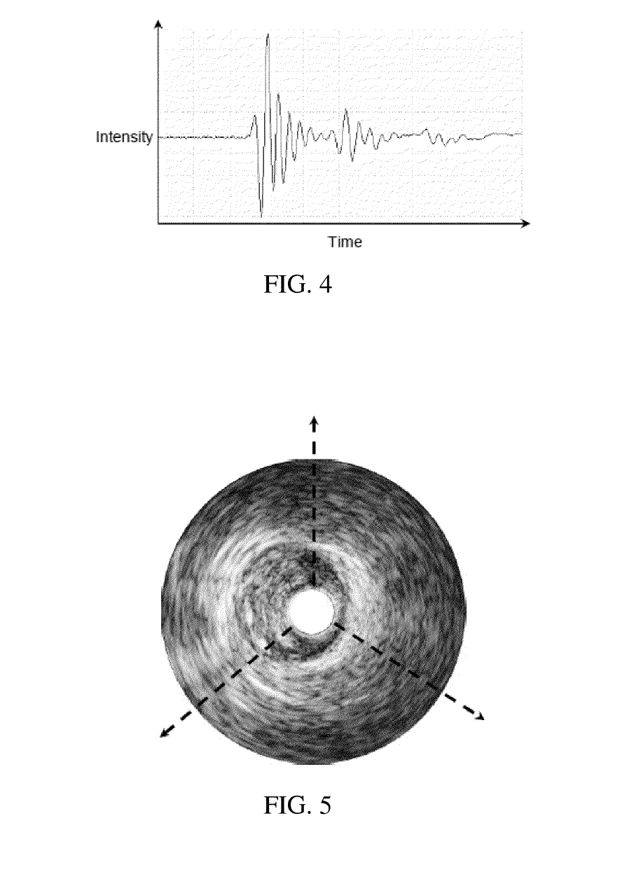

The same transducer elements can be used to acquire different types of intravascular data, such as flow data, motion data and structural image data. The different types of intravascular data are acquired based on different manners of operation of the transducer elements. For example, in gray-scale imaging mode, the transducer elements transmit in a certain sequence with apertures of 14 elements being active at any given time, in which one out of those fourteen elements sends out an ultrasound pulse and the remaining elements receive or transduce the ultrasound back (FIG. 3). In this respect, the transmit-receive sequence moves around all 64 elements to create a total of 896 transmit-receive sequences which are then post-processed through a synthetic aperture focusing to create 256 or 512 or other pre-determined number of individual scan lines that are scan-converted to create one gray-scale IVUS image (FIGS. 4-5). FIG. 4 depicts a typical individual scan line with Time on the x-axis that corresponds to distance from the catheter into the vessel lumen and wall. FIG. 5 depicts a scan-converted and log-compressed gray-scale IVUS image constructed from 256 scan-lines. Each of the dotted lines on the image represents one scan line. Methods for constructing IVUS images are well-known in the art, and are described, for example in Hancock et al. (U.S. Pat. No. 8,187,191), Nair et al. (U.S. Pat. No. 7,074,188), and Vince et al. (U.S. U.S. Pat. No. 6,200,268), the content of each of which is incorporated by reference herein in its entirety.

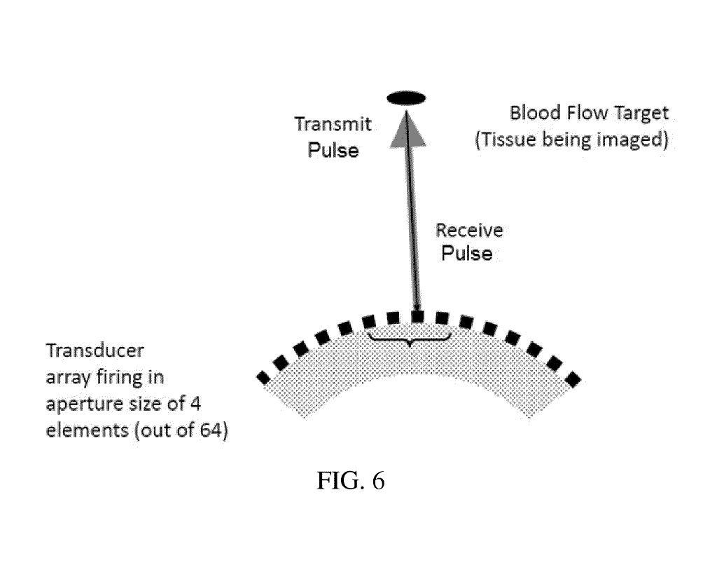

In flow imaging mode, the IVUS catheter transmit sequence is changed so that first the transducers operate as usual to acquire the gray-scale image scan-lines and then the transducer elements are operated in a different way to collect the information on the motion or flow. FIG. 6 illustrates the transducer operating sequence in the flow imaging mode. The acoustic information is acquired by transmitting on four adjacent transducer elements simultaneously and then receiving on the same four simultaneously.

This sequence is repeated 64 times on the same four elements, producing 64 A scan-lines of data from the same physical beam location, which are then averaged by two, resulting in 32 lines. The sequence is stepped around each transducer element, i.e., moves over one transducer element to form the next set of four elements as the next aperture. The transmit-receive process is repeated on the next set of four elements. This process enables one image (or frame) of flow data to be acquired. The acquisition of each flow frame of data is interlaced with an IVUS gray scale frame of data. Operating an IVUS catheter to acquire flow data and constructing images of that data is further described in O'Donnell et al. (U.S. Pat. No. 5,921,931), U.S. Provisional Patent Application No. 61/587,834, and U.S. Provisional Patent Application No. 61/646,080, the content of each of which is incorporated by reference herein its entirety.

Commercially available fluid flow display software for operating an IVUS catheter in flow mode and displaying flow data is CHROMAFLO (IVUS fluid flow display software; Volcano Corporation).

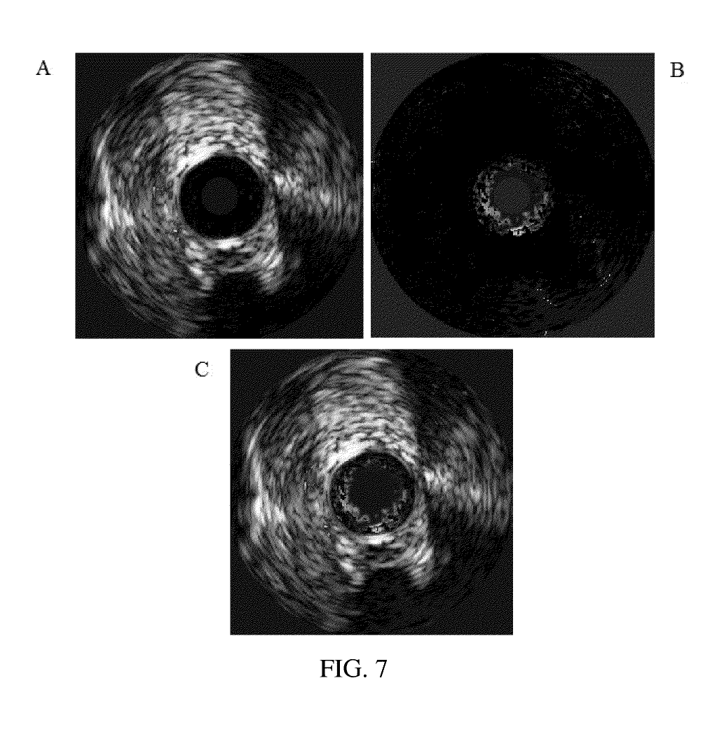

As shown in FIG. 7, panels A-C, the flow data can be overlaid with the image data to provide a combined image. FIG. 7 depicts a 360 degree cross-section view of the inside of a vessel and therefore the flow data represents blood flow in the vessel. The combined image provides an additional level of detail to a physician that is not provided by any IVUS image alone. Panel A shows a gray-scale image alone. Panel B shows an image of flow data alone. Panel C shows an overlay of the image of the flow data on the gray-scale image. It should be appreciated that blood-vessel data can be used in a number of applications including, but not limited to, diagnosing and/or treating patients. For example, blood-vessel data can be used to identify and/or image blood vessel borders or boundaries, as provided by U.S. Pat. No. 6,381,350, which is incorporated by reference herein in its entirety. Another use for blood-vessel data is for classifying and/or imaging vascular plaque, as provided by U.S. Pat. No. 6,200,268, which is also incorporated by reference herein in its entirety. Another use for blood-vessel data is to classify vascular tissue, as provided by U.S. Pat. No. 8,449,465, which is also incorporated by reference herein in its entirety.

The rate at which data can be acquired effects the resolution of the image. The acquisition rate of data (frames per second) is affected by the field-of-view. For example, for IVUS catheters that operate at 20 MHz frequency and that use a 20 mm field-of-view for the user, hence 10 mm radially in each direction from the catheter/transducers, the acquisition frame rate is about 30 frames per second. For IVUS catheters that operate at 20 MHz frequency and that use a 24 mm field-of-view for the user, the acquisition frame rate is about 24 frames per second. For IVUS catheters that operate at 10 MHz frequency and use a 60 mm field-of-view for the user, the acquisition frame rate is about 11 frames per second.

A problem that occurs when a single set of transducers is used to collect a plurality of different types of data is a drop in acquisition frame rate for each type of data collected, for example, a 50% or more reduction in acquisition frame rate from single mode operation to multi-mode operation. Using the 20 MHz frequency 20 mm field-of-view catheter as an example, the gray-scale acquisition frame rate is about 30 frames per second. However, when the transducers are used to acquire image and flow data, the acquisition frame rate for the composite image data drops from 30 frames per second to about 14 frames-per-second (e.g., 12-15 frames-per-second), owing to the additional data being collected alternatively, first in the image data firing mode and then the flow data firing mode.

Regardless of the frequency or the field-of-view, causing a single set of transducers to acquire more than one type of data results in a drop in the overall acquisition frame rate. This is because the transducers are limited by the time for the ultrasound echo to return to the transducer from the field of view. For example, with IVUS catheters that operate at 20 MHz frequency and that use a 24 mm field-of-view, the acquisition frame rate drops from about 24 frames per second to about 9-10 frames per second. For IVUS catheters that operate at 10 MHz frequency and that use a 60 mm field-of-view, the acquisition frame rate drops from about 11 frames per second to about 4 frames per second.

The invention provides various techniques to modify the transducer firing sequence for at least one of the types of data that is acquired by the single set of transducers. In that manner, the acquisition frame rate for that type of data is increased and the overall acquisition frame rate (for all of the types of data being acquired) is increased. Accordingly, adjusting the acquisition frame rate for a single type of data compensates for the drop-off in the acquisition frame rates of the other types of data, thereby increasing the acquisition frame rates of the other types of data without adjusting the manner in which the transducers operate to acquire that type of data.

The following embodiments are described using a combination of image data and flow data, and are discussed in the context of changing the acquisition frame rate for the flow data. However, it will be appreciated that the methods and systems described herein apply to more than just two types of data acquired by a single set of transducers, for example three types of data, four types of data, five types of data, ten types of data, etc. Additionally, it will also be appreciated that it does not have to be the flow data that is adjusted. The acquisition of the image data can be adjusted without changing the acquisition of the flow data. In other embodiments, adjustments can be made for acquisition of more than one type of data, e.g., adjustments can be made for the acquisition of both the image data and the flow data.

In one embodiment, the field-of-view for one type of data is limited or narrowed as compared to the other type of data. As discussed above, the transducers are operated through a series of transmit/receive modes, that being a single transducer sends a signal in transmit mode and then receives the signal in receive mode, which represents one sequence. The transducer does not switch back to transmit mode until the previously transmitted signal is received. By narrowing the field-of-view, each transmit/receive sequence is shortened. Shortening each transmit/receive sequence allows for faster acquisition of the flow data, allowing the transducers to more quickly switch from acquiring flow data to acquiring image data, thereby increasing the acquisition frame rate for not only the flow data, but also the composite image data. For example, for an IVUS catheter that operates at 10 MHz frequency, the transducers would be operated in a manner that allows image data to be acquired at a 60 mm field-of-view. However, for the flow data, the field-of-view would be limited or narrowed from 60 mm, to a smaller field-of-view, such as 50 mm, 40 mm, 30 mm, or a lower number or any number between the exemplified numbers.

Specifically, narrowing the field-of-view of the transducers means that in transmit mode, the transducers take the same time to transmit the ultrasound pulse, however, take less time to receive the transmitted pulse, because a smaller area of tissue closer to the transducer is being interrogated. With each transmit/receive cycle being shorter, it takes less time between cycles, and consequently less time to acquire the flow data. That consequently allows the transducers to more quickly switch from acquisition of flow data to acquisition of image data, thereby increasing the acquisition frame rate for the final composite image data. In this embodiment, the acquisition frame rate for the flow data has been increased, and because the transducers spend less time acquiring the flow data, the acquisition frame rate for the composite image data increases, without having to make adjustments or modifications for the acquisition of the image data. It is possible for this technique to be used with the image data instead of the flow data, or for this technique to be applied to both the image and the flow data.

In another embodiment, the manner of operation of the transducers is adjusted for one type of data to lower the resolution of the flow-data acquired. Typically, the flow data is acquired by operating four adjacent transducer elements simultaneously and then receiving on the same four simultaneously. This sequence is repeated 64 times on the same four elements, producing 32 averaged A scan-lines of data from the same physical beam location. The sequence is stepped around each transducer element, i.e., moves over one transducer element to form the next set of four elements. The process is repeated on the next set of four elements. To lower the resolution, the firing sequence is adjusted so that the sequence skips a transducer when forming a new set, i.e., move two or more transducers from the previous set of four to form the next set of four transducers. For example, a new operating sequence would be operating four adjacent transducer elements simultaneously and then receiving on the same four simultaneously. This sequence is repeated 64 times on the same four elements, producing 32 averaged A scan-lines of data from the same physical beam location. The sequence skips over one or more transducer elements, skipping at least one element, and forms the next set of four transducer elements. The process is repeated on the next set of four elements. Decreasing the number of sets allows for faster completion of the cycle around the catheter body, which results in faster acquisition of the flow data. That allows the transducers to more quickly switch from acquiring flow data to acquiring image data, thereby increasing the acquisition frame rate for not only the flow data, but also the image data. Stated another way, in this embodiment, the acquisition frame rate for the flow data has been increased, and because the transducers spend less time acquiring the flow data, the acquisition frame rate for the image data increases, without having to make adjustments or modifications for the acquisition of the image data. It is possible for this technique to be used with the image data instead of the flow data, or for this technique to be applied to both the image and the flow data.

In another embodiment, the acquisition rate of the flow data can be improved by lowering the signal-to-noise ratio (SNR) of the flow-data by acquiring less data. Typically, the flow data is acquired by operating four adjacent transducer elements simultaneously and then receiving on the same four simultaneously. This sequence is repeated 64 times on the same four elements, producing 32 averaged A scan-lines of data from the same physical beam location. The sequence is stepped around each transducer element, i.e., moves over one transducer element to form the next set of four elements. The process is repeated on the next set of four elements.

To lower the SNR, the transmit sequence is adjusted so that the transmit is repeated only 32 times, instead of 64 times, and there is no averaging. In this approach, 32 A scan-lines of flow data are still acquired for the same physical beam location. Decreasing the number of transmit pulses for each set of transducers allows for faster acquisition at each set, and therefore results in faster completion of the cycle around the catheter body, which results in faster acquisition of the flow data. That allows the transducers to more quickly switch from acquiring flow data to acquiring image data, thereby increasing the acquisition frame rate for not only the flow data, but also the composite image data. Stated another way, in this embodiment, the acquisition frame rate for the flow data has been increased, and because the transducers spend less time acquiring the flow data, the acquisition frame rate for the image data increases, without having to make adjustments or modifications for the acquisition of the structural image data. It is possible for this technique to be used with the image data instead of the flow data, or for this technique to be applied to both the image and the flow data.

In another embodiment, a sectors approach is applied. In this manner, only a subset of transducers, corresponding to an area of interest are used to produce the flow data. This embodiment is accomplished by generating a 360° gray-scale cross-section image of the vessel of interest. From that image, a certain subsection is chosen for which flow data will be generated. In that manner, only a subset of all of the transducers need to be used to acquire flow data that corresponds to the subsection of interest. Accordingly, the acquisition frame rate for the flow data has been increased, and because the transducers spend less time acquiring the flow data, the acquisition frame rate for the composite image data increases, without having to make adjustments or modifications for the acquisition of the image data. It is possible for this technique to be used with the image data instead of the flow data, or for this technique to be applied to both the image and the flow data.

The subsection can be any subsection of the 360° image, e.g., a 45° subsection, a 60° subsection, a 90° subsection, a 120° subsection, a 135° subsection, a 180° subsection, a 225° subsection, a 240° subsection, a 270° subsection, a 300° subsection, or a 315° subsection. In certain embodiments, more than one subsection is selected at any one point in time, such that flow data for a plurality of sections is generated simultaneously. The subsections can be adjacent each other or can be disconnected from each other.

The subsections can be selected manually by a user highlighting a section of the 360° image in which flow data should be displayed. This could be done by a simple interactive mode between the user and the GUI by use of an interactive device like a computer mouse, or a touch-screen monitor to communicate to the software which would then govern adjusting the subtended-sector of interest and translating that information to the imaging boards of the IVUS system which define the transducer operation. Alternatively, the flow data can automatically be displayed to subsections. In certain embodiments, the flow data is automatically split into 120° subsections that a user can toggle through with respect to the 360° image, using a simple GUI control or a dedicated control on the IVUS system keyboard, control pad or touch-screen control pad. In that manner, flow data is overlaid with one-third of the 360° image at any one time. It is possible for this technique to be used with the image data instead of the flow data, or for this technique to be applied to both the image and the flow data.

Any of the above embodiments may be combined to increase the overall acquisition frame rate when a single set of transducers is being used to acquire a plurality of different types of date. For example, the low resolution embodiment above can be applied to the image data to produce a 360° low resolution image of a vessel. For that to be performed, one or more of the transducers would be skipped when forming a next set of 14 transducer elements from the previous set of 14 transducer elements. From that low resolution image, a subsection of the image can be selected to be overlaid with flow data. In that manner, both the manner of operation of the transducers is being adjusted from both the image data and the flow data.

Another example could be to first produce the low resolution composite image and then to automatically detect, or semi-automatically detect the area of interest on the 360 degree composite image, with user input. The system could then automatically switch from the low resolution composite image mode to the sector composite image mode to highlight the sector-of-interest in the final composite image.

In preferred embodiments, the above described techniques are applied to 10 MHz, 60 mm field-of-view IVUS catheters where an image acquisition rate of 4-5 frames per second is not sufficient to image flow within the peripheral vasculature. Increasing the acquisition frame rate, for example to about 9 frames per second, which is accomplished by systems and methods of the invention, allows a 10 MHz, 60 mm filed-of-view IVUS catheter to be used to display image and flow data within the peripheral vasculature.

A particular advantage is to allow flow detection with 10 MHz phased array devices. This is useful for physicians performing peripheral vascular procedures in detecting thrombi, dissections, and endoleaks in the stent grafting procedures for treating abdominal aortic aneurysms (AAA). Also, with the use of a flow detection capability, users could be more easily guided through endovascular aneurysm/aortic repair (EVAR) like procedures and thus reduce the contrast dosage to patients due to the angiographic imaging techniques that are currently employed in the catheterization and vascular surgery laboratories. More specifically, flow detection capabilities with a 10 MHz device would make it easier to detect: endoleaks, dissections, thrombus, stent apposition, and results of interventional procedures to restore normal blood flow to the vasculature.

In another embodiment, the invention provides systems and methods that allow for controlled variable frame rates. Any of the above approaches can be used to achieve controlled variable frame rates. In the variable frame rate approach, the transducers are operated to acquire a single type of data at a standard acquisition frame rate for that type of data. At a user's request, the manner of operation of the transducers can be adjusted so that the transducers can acquire more than one type of data for a location in a vessel. Upon signaling to the system to acquire more than one type of data, the embodiments described above are used so that the composite data, e.g., structural image and/or flow data, are acquired with adjusted acquisition frame rates. In this manner, a single data set for a physical structure includes the same type of data, acquired at two different acquisition frame rates.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A system for producing an intravascular image, the system comprising:
a central processing unit (CPU); and
storage coupled to the CPU for storing instructions that when executed by the CPU cause the CPU to:
cause transducers of an intravascular ultrasound device to generate a plurality of different types of data, each type of data being based on a different manner of operation of the transducers;
adjust the manner of operation of the transducers for one of the types of data to thereby adjust an acquisition frame rate of the one type of data to an adjusted acquisition frame rate;
receive the one type of data at the adjusted acquisition frame rate; and
display an intravascular image that comprises the one type of data at the adjusted acquisition frame rate.

2. The system according to claim 1, wherein the adjust instructions that when executed by the CPU cause the CPU to: decrease field-of-view of the transducers for the one type of data.

3. The system according to claim 1, wherein the adjust instructions that when executed by the CPU cause the CPU to: revise the manner of operation of the transducers to lower the acquisition resolution for the one type of data.

4. The system according to claim 1, wherein the adjust instructions that when executed by the CPU cause the CPU to: select a subset of the transducers to be used to acquire the one type of data from a sub-region of a vessel.

5. The system according to claim 1, wherein the adjust instructions that when executed by the CPU cause the CPU to:
use the transducers to produce a 360° intravascular image;
display the 360° intravascular image;
select an area-of-interest within the image; and
select a subset of the transducers to acquire the one type of data in the area-of-interest.

6. The system according to claim 1, wherein the adjust instructions that when executed by the CPU cause the CPU to:
use the transducers to produce a 360° intravascular image;
display the 360° intravascular image;
select multiple areas-of-interest within the image;
select multiple subsets of transducers, each sub-set of transducers corresponding to a selected area-of interest; and
use the sub-sets of transducers to acquire the one type of data in each of the areas-of-interests.

7. The system according to claim 1, wherein the image further comprises at least one other type of data.

8. The system according to claim 7, wherein a first type of data is intravascular structural image data and a second type of data is intravascular flow data.

9. The system according to claim 8, wherein the intravascular flow data is the one type of data that is adjusted.

10. The system according to claim 8, wherein the intravascular structural image data is the one type of data that is adjusted.

11. A method for producing an intravascular image, the method comprising:
causing transducers of an intravascular ultrasound device to generate a plurality of different types of data, each type of data being based on a different manner of operation of the transducers;
adjusting the manner of operation of the transducers for one of the types of data to thereby adjust an acquisition frame rate of the one type of data to an adjusted acquisition frame rate;
receiving the one type of data at the adjusted acquisition frame rate; and
displaying an intravascular image that comprises the one type of data at the adjusted acquisition frame rate.

12. The method according to claim 11, wherein adjusting comprises decreasing field-of-view of the transducers of the one type of data.

13. The method according to claim 11, wherein adjusting comprises revising the manner of operation of the transducers to lower the acquisition resolution of the one type of data.

14. The method according to claim 11, wherein adjusting comprises selecting a sub-set of the transducers to be used to acquire the one type of data in a sub-region of a vessel.

15. The method according to claim 11, wherein adjusting comprises:
using the transducers to produce a 360° intravascular image;
displaying the 360° intravascular image;
selecting an area-of-interest within the image; and
selecting a sub-set of the transducers to acquire the one type of data in the area-of-interest.

16. The method according to claim 11, wherein adjusting comprises:
using the transducers to produce a 360° intravascular image;
displaying the 360° intravascular image;
selecting multiple areas-of-interest within the image;
selecting multiple sub-sets of transducers, each sub-set of transducers corresponding to a selected area-of interest; and
using the sub-sets of transducers to acquire the one type of data in each of the areas-of-interests.

17. The method according to claim 11, wherein the image further comprises at least one other type of data.

18. The method according to claim 17, wherein a first type of data is intravascular structural image data and a second type of data is intravascular flow data.

19. The method according to claim 18, wherein the intravascular flow data is the one type of data that is adjusted.

20. The method according to claim 18, wherein the intravascular structural image data is the one type of data that is adjusted.
